(19) United States
(12) Patent Application Publication
(10) Pub. No.: US 2022/0241102 A1
(43) Pub. Date: Aug. 4, 2022

(54) FEEDBACK-CONTROLLED PRESSURE MONITORING SYSTEM FOR LIMB-STABILIZING MEDICAL PRESSURE SPLINTS

(71) Applicant: University of Washington, Seattle, WA (US)

(72) Inventors: Jason Germany, Seattle, WA (US); Abhijith Shasheendra, Seattle, WA (US); Shahram Aarabi, Seattle, WA (US)

(73) Assignee: University of Washington, Seattle, WA (US)

(21) Appl. No.: 17/619,238

(22) PCT Filed: Jun. 11, 2020
(86) PCT No.: PCT/US2020/037153

§ 371 Date: Dec. 14, 2021

Related U.S. Application Data
(60) Provisional application No. 62/862,288, filed on Jun. 17, 2019.

(51) Int. Cl. A61F 5/058; A61F 7/00

(52) U.S. Cl. CPC A61F 5/05816; A61F 2007/0056; A61F 7/00; A61F 5/05833

(57) ABSTRACT

Feedback-controlled pressure monitoring system for limb-stabilizing medical pressure splints are described herein. In one embodiment, a limb-stabilizing system, includes: a pressure splint; a pressure sensor operatively coupled with the pressure splint; a controller configured to receive a first signal from the pressure sensor and to send a second signal to a pump; and the pump operatively coupled to the pressure splint. The pump is configured to adjust a pressure of air inside the pressure splint based on the second signal received from the controller.

CROSS-REFERENCE(S) TO RELATED APPLICATION(S)

This application claims the benefit of Provisional Application No. 62/862,288, filed Jun. 17, 2019, which is incorporated herein by reference.

### BACKGROUND

Management of a variety of musculoskeletal conditions requires the use of a cast or splint. Splints are generally understood as immobilizers that can accommodate swelling. This attribute makes splints useful for managing a variety of acute musculoskeletal conditions in which swelling is anticipated, such as acute fractures or sprains, or for initial stabilization of reduced, displaced, or unstable fractures before orthopedic intervention.

In contrast with splints, casts are circumferential immobilizers. Therefore, casts provide superior immobilization, but are less forgiving, have higher complication rates, and are generally reserved for complex and/or definitive fracture management.

Selection of a specific cast or splint varies based on the area of the body being treated, and on the acuity and stability of the injury. To maximize benefits while minimizing complications, the use of casts and splints is generally short term. However, even when using a splint for a relatively short time (for example, several hours) the condition of the immobilized limb may sufficiently change through, for example, swelling, different outside pressure, position of the patient body, etc., to make the splints uncomfortable to the patient. Accordingly, systems and methods are needed for making splints more comfortable and/or more functional as the conditions affecting the patient change.

### SUMMARY

This summary is provided to introduce a selection of concepts in a simplified form that are further described below in the Detailed Description. This summary is not intended to identify key features of the claimed subject matter.

Briefly, the inventive technology is directed to air splints that sense pressure inside the splint and regulate the pressure to stabilize the splint. In some embodiments, the splint includes a feedback loop for regulating the pressure to a predetermined target point. Such feedback loops may operate in real time to increase/decrease pressure in response to fluid leaking out of or into the splint, limb swelling, changes in ambient pressure or temperature, etc. In some embodiments, a pressure in the splint is maintained by a 2-way pump. The inventive technology may be suitable for pressurized splints and for vacuum-based splints.

### DESCRIPTION OF THE DRAWINGS

The foregoing aspects and the attendant advantages of the inventive technology will become more readily appreciated as the same are understood with reference to the following detailed description, when taken in conjunction with the accompanying drawings, wherein:

FIG. 1 is a partially schematic drawing of an air splint in accordance with an embodiment of the present technology;

FIG. 2 is a partially schematic drawing of a vacuum splint in accordance with an embodiment of the present technology;

FIG. 3 is an isometric drawing of a pump in accordance with an embodiment of the present technology;

FIG. 4 is a schematic diagram of a system in accordance with an embodiment of the present technology; and

FIG. 5 is a flowchart of a method for operating a splint in accordance with an embodiment of the present technology.

### DETAILED DESCRIPTION

While several embodiments have been illustrated and described, it will be appreciated that various changes can be made therein without departing from the spirit and scope of the claimed subject matter.

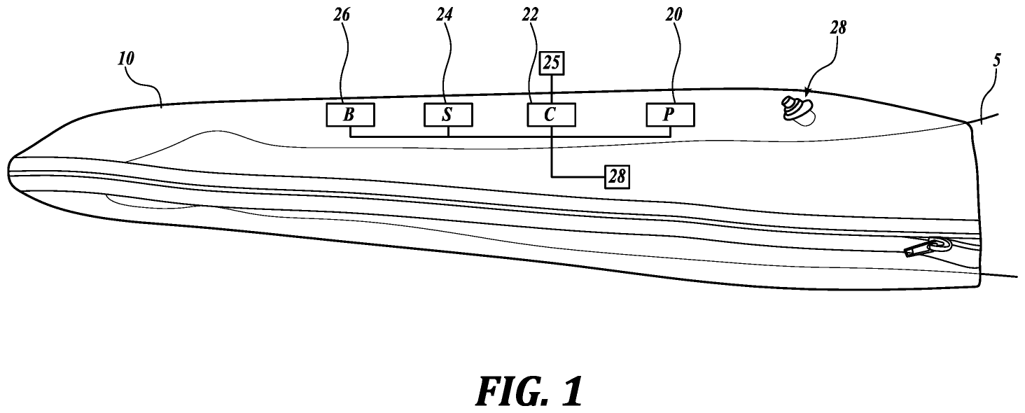

FIG. 1 is a partially schematic drawing of an air splint 10 in accordance with an embodiment of the present technology. The air splint 10 may partially or completely house a patient's limb 5. When properly inflated, the air splint 10 supports and immobilizes the limb 5.

In some embodiments, the air splint 10 includes a pump 20 that is configured to add or remove air from the air splint 10. The operation of the pump 20 (e.g., turning the pump and off) may be controlled by a controller 22 based on, at least in part, sensing the pressure inside the splint by a pressure sensor 24. In some embodiments, proper functioning of the sensor 24 may be verified by comparing a pressure readout of this sensor to a pressure readout of an ambient pressure sensor 25. For example, such a verification may take place before the air splint 10 is pressurized since both sensors 25 and 28 should read close to atmospheric pressure at such time. In some embodiments, the air splint includes a relief valve 28 (e.g., a safety valve) that depressurizes the air splint 10 when a predetermined pressure threshold is reached. In different embodiments, the above described elements of the limb support system may be carried by the air splint itself or may be configured separately from the air splint 10. Furthermore, the pump 20, controller 22 and/or pressure sensors 24, 25 may be energized by a source of power 26. In some embodiments, the source of power 26 is a battery.

In operation, the air splint 10 may lose pressure or may become over pressurized. In response to the pressure inside the air splint 10 falling outside of a predetermined range, the pressure sensor 24 provides pressure signal to the controller 22, which in turn may activate the pump 20 to bring the pressure back within the predetermined range. The communication between the controller 22 and the pressure sensors 24, 25 and the pump 20 may be through conductive wires or wireless (e.g., Bluetooth or other wireless communication).

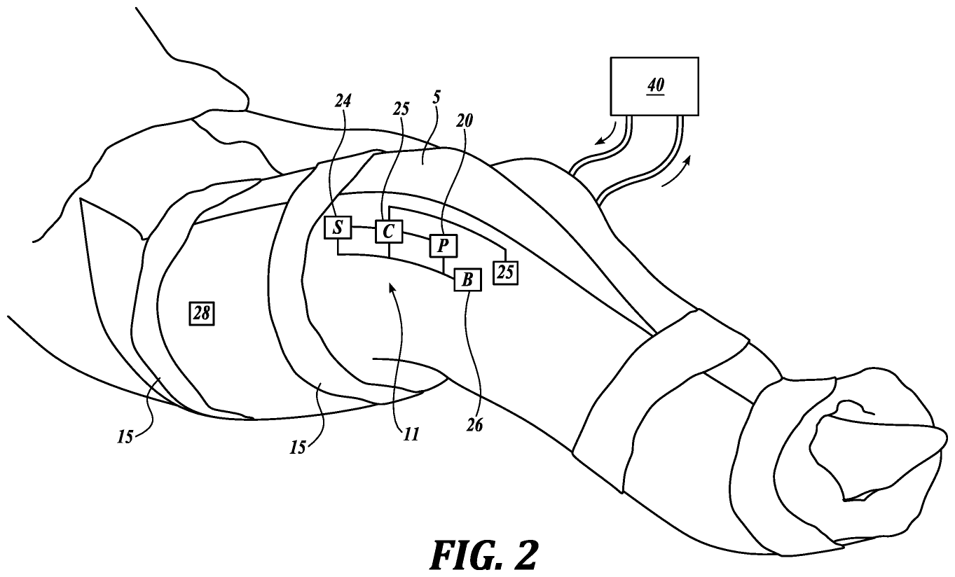

FIG. 2 is a partially schematic drawing of a vacuum splint 11 in accordance with an embodiment of the present technology. In the context of this specification, the vacuum splint 11 and the air splint 10 are collectively referred to as pressure splints. In operation, the vacuum splint 11 is maintained at a below-atmospheric pressure, and the air splint 10 is maintained at an above-atmospheric pressure.

The vacuum splint 11 may contain stiffening elements 15 that add stiffness to the splint and help to immobilize the limb. In some embodiments, the stiffening elements 15 may be configured inside the vacuum splint to stabilize or give shape to the vacuum splint.

Some non-exclusive examples of such stiffening elements are rigid sticks or other shapes, granular material (e.g., sand), hemming at the vacuum splint edges, etc. The illustrated vacuum splint 11 partially encloses the limb, however, in other embodiments patient's limb may be completely enclosed.

In operation, the pressure (vacuum) inside the vacuum splint 11 may drift away from its predetermined range. Analogously to the operation of the air splint 10, the pressure sensor 24 provides pressure signal to the controller 22, which in turn may activate the pump 20 to bring the pressure back within the predetermined range.

In some embodiments, a chiller 40 can cool the patient's limb while being immobilized inside the vacuum splint 11 or air splint 10. In different embodiments, the chiller 40 may be attached to the pressure splint by suitable coolant hoses, or the chiller may be directly attached to the pressure splint.

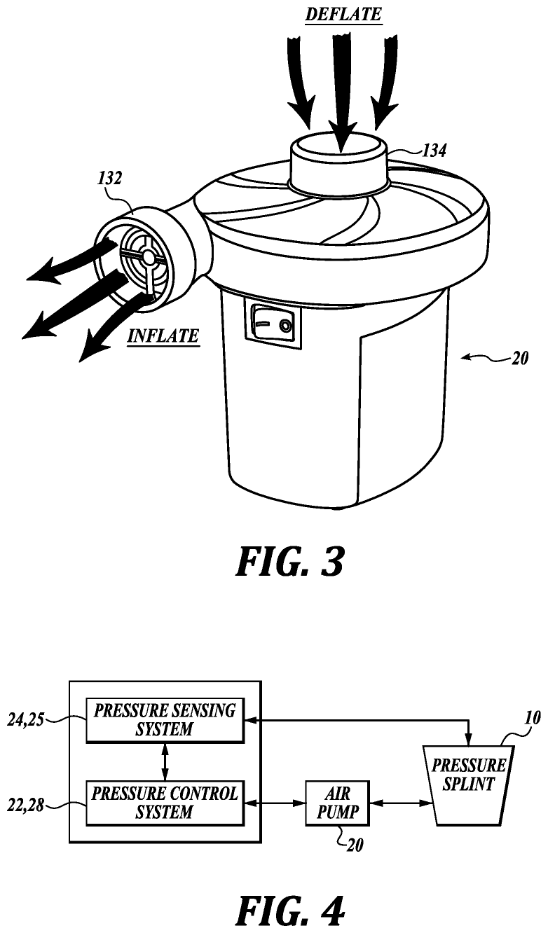

FIG. 3 is an isometric drawing of a pump 20 in accordance with an embodiment of the present technology. In some embodiments, the pump 20 is a 2-way pump that can pressurize the attached pressure splint through an outlet 132 or depressurize the pressure splint through an inlet 134. The operation of the pump 20 may be controlled by a controller. In some embodiments, the pressure sensor 24, the controller 22 and the source of power 26 may be integrated into the pump 20. In different embodiments, the pump 20 may be a centrifugal pump or a volumetric pump.

FIG. 4 is a schematic diagram of a system in accordance with an embodiment of the present technology. In operation, the pressure sensing system (e.g., the pressure sensor 24) senses the pressure inside the pressure splint (e.g., air splint 10 or vacuum splint 11). The pressure control system (e.g., the controller 22) receives input signal from the pressure sensing system, and in turn provides signal to the air pump to turn on or off. Based on this feedback-controlled pressure monitoring, pressure inside the pressure splint is maintained within a predetermined range, therefore maintaining required limb immobility and comfort of the patient. In some embodiment the controller 22 may an Arduino board Mega 2560. In some embodiments, the pressure sensors 24, 25 may be piezoresistive transducers, for example Honeywell ABP MPxx5010 transducers.

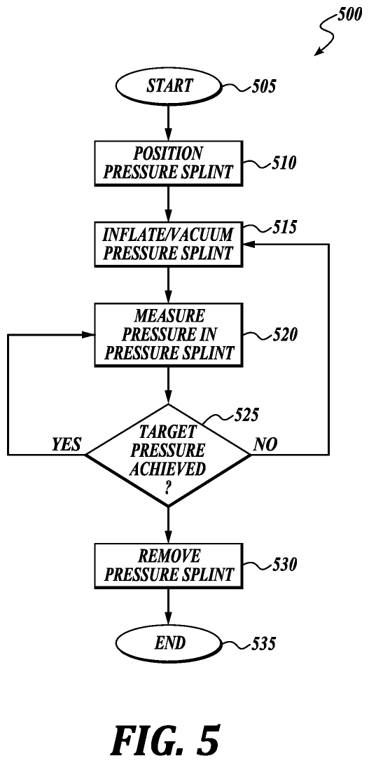

FIG. 5 is a flowchart of a method 500 for operating a splint in accordance with an embodiment of the present technology. In some embodiments, the method may include additional steps or may be practiced without all steps illustrated in the flow chart.

The method starts in block 505. In block 510, the pressure splint is positioned over the limb that requires immobilization. In block 515, the pressure splint is inflated or vacuumed to its initial set point by, for example, the pump 20. In block 520, pressure in the pressure splint is measured by, for example, a pressure sensor 24. The pressure measurement data may be provided to the controller 22.

In block 525, the controller 22 ascertains whether the target pressure is achieved. The target pressure may correspond to a predetermined pressure range or to a single pressure value. If the target pressure is achieved, the pressure inside the pressure splint is measured again in block 520. If the target pressure is not achieved, that is, the measured pressure is outside the predetermined pressure range, the controller activates the pump in block 515. For example, if the measured pressure is below the target pressure, the pump 20 may add air into the pressure splint thus, for example, further increasing pressure inside the air splint. Conversely, if the measured pressure is above the target pressure, the pump 20 may remove air from the pressure splint, thus, for example, further increasing vacuum inside the vacuum splint.

In block 530, the pressure splint is removed. The method ends in block 535.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” etc., mean plus or minus 5% of the stated value.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

What is claimed is:

1. A limb-stabilizing system, comprising:
a pressure splint;
a pressure sensor operatively coupled with the pressure splint;
a controller configured to receive a first signal from the pressure sensor and to send a second signal to a pump; and
the pump operatively coupled to the pressure splint, wherein the pump is configured to adjust a pressure of air inside the pressure splint based on the second signal received from the controller.

2. The system of claim 1, wherein the pressure splint is an air splint configured to maintain an above-atmospheric pressure inside the air splint.

3. The system of claim 2, wherein the pump is configured to increase the pressure in the air splint when the pressure of air inside the air splint falls below a predetermined range.

4. The system of claim 1, wherein the pressure splint is a vacuum splint configured to maintain a below-atmospheric pressure inside the vacuum splint.

5. The system of claim 4, wherein the pump is configured to decrease the pressure in the vacuum splint when the pressure of air inside the vacuum splint increases above a predetermined range.

6. The system of claim 1, wherein the source of air is a 2-way air pump configured to increase the pressure in the pressure splint by providing air to the pressure splint or to decrease the pressure in the pressure splint by evacuating air from the pressure splint.

7. The system of claim 1, wherein the pressure sensor is a first pressure sensor, the system further comprising a second pressure sensor that is an ambient pressure sensor operationally coupled to the controller, wherein the controller is configured to verify accuracy of the first pressure sensor by comparing readings between the first pressure sensor and the second pressure sensor.

8. The system of claim 1, further comprising a relief valve configured to release an excess pressure from the pressure splint.

9. The system of claim 1, further comprising a chiller configured to cool a limb under treatment.

10. A method for stabilizing a limb, comprising:
positioning a pressure splint over the limb;
pressurizing the pressure splint to a target pressure range;
monitoring a pressure inside the pressure splint by a pressure sensor that is operationally coupled to a controller; and
if the pressure is outside of a target pressure range, activating a pump by the controller to adjust the pressure in the pressure splint.

11. The method of claim 10 wherein the source of air is a 2-way air pump configured to increase pressure in the pressure splint by providing air to the pressure splint or to decrease pressure in the pressure splint by evacuating air from the air splint.

12. The method of claim 11, wherein the pressure splint is an air splint configured to maintain an above-atmospheric pressure inside the air splint.

13. The method of claim 12, wherein the pump is configured to increase pressure in the air splint when the pressure of air inside the air splint falls below the target pressure range.

14. The method of claim 11, wherein the pressure splint is a vacuum splint configured to maintain a below-atmospheric pressure inside the vacuum splint.

15. The method of claim 14, wherein the pump is configured to decrease pressure in the vacuum splint when the pressure of air inside the vacuum splint increases above the target pressure range.

16. The method of claim 10, further comprising cooling the limb by a chiller.

17. A computer-readable storage device storing non-volatile computer-executable instructions, which, when executed, cause pressurization or depressurization of a pressure splint by:
monitoring a pressure inside the pressure splint by a pressure sensor that is operationally coupled to a controller; and
if the pressure is outside of a target pressure range, activating a pump by the controller to adjust the pressure in the pressure splint.

18. The computer-readable storage device of claim 17, wherein the pressure sensor is a first pressure sensor, the computer-readable storage device further comprising instructions for:
verifying accuracy of the first pressure sensor by comparing readings between the first pressure sensor and a second pressure sensor that is an ambient pressure sensor.

19. The computer-readable storage device of claim 18, wherein the first pressure sensor and the second pressure sensor are operatively coupled to a controller.

20. The computer-readable storage device of claim 17, wherein the pump is a 2-way air pump configured to increase the pressure in the pressure splint by providing air to the air splint or to decrease the pressure in the pressure splint by evacuating air from the air splint.
